(19) United States
(12) Patent Application Publication
(10) Pub. No.: US 2021/0000782 A1
(43) Pub. Date: Jan. 7, 2021

(54) APPLICATION OF DROFENINE OR SALT THEREOF TO PREPARATION OF MEDICINE FOR TREATING PERIPHERAL NEURON AXONAL INJURY AND PERIPHERAL RELATED NEUROPATHY

(71) Applicant: NANJING UNIVERSITY OF CHINESE MEDICINE, Jiangsu (CN)

(72) Inventors: Xu SHEN, Jiangsu (CN); Jiaying WANG, Jiangsu (CN); Xiaoju XU, Jiangsu (CN)

(73) Assignee: NANJING UNIVERSITY OF CHINESE MEDICINE, Jiangsu (CN)

(21) Appl. No.: 16/980,429

(22) PCT Filed: Jan. 14, 2019
(86) PCT No.: PCT/CN2019/071557

§ 371 Date: Sep. 14, 2020

(30) Foreign Application Priority Data
Dec. 25, 2018 (CN) 201811589167.0

(51) Int. Cl. A61K 31/216; A61P 25/02

(52) U.S. Cl. CPC A61K 31/216; A61P 25/02

(57) ABSTRACT

The present invention relates to application of drofenine or a pharmaceutically acceptable salt thereof in the preparation of medicine for treating peripheral neuron axonal injury and peripheral related neuropathy. The drofenine or the pharmaceutically acceptable salt thereof has a significant effect on promoting growth of peripheral sensory neurites, and can relieve symptoms of low nerve conduction velocity and sensation loss of STZ-induced type I diabetic mice and db/db type II diabetic mice, thereby treating diabetic peripheral neuropathy.

### BACKGROUND

### Technical Field

The present invention relates to the technical field of medicine, particularly to application of drofenine or a pharmaceutical composition thereof to preparation of medicine for treating diabetic peripheral neuropathy, and more particularly to application of drofenine or a pharmaceutically acceptable salt or a pharmaceutical composition thereof in preparation of medicine for treating peripheral neuropathy such as the diabetic peripheral neuropathy due to its protection effect on peripheral sensory neurons.

### Description of Related Art

Diabetic peripheral neuropathy (DPN) refers to symptoms related to peripheral nerve dysfunction of a diabetic patient when other causes are excluded. Clinical features are symmetrical pain and paresthesia, and symptoms of lower limbs are more common than those of upper limbs. The paresthesia includes numbness, formication, insect infestation, heat and electric shock-like sensations, often going up from distal toes to knees, and patients have sock and glove wearing type sensations. Lower limb arthropathy and ulcers may occur in patients with serious sensory disorders. The pain includes stabbing pain, burning pain and drilling pain, seems to appear in deep bone marrow, is sometimes as severe as amputation pain, and is light in daytime and serious at night. When motor nerves are involved, muscle strength often decreases in different degrees, and dystrophic muscular atrophy occurs in a late stage. Peripheral neuropathy may be bilateral, unilateral, symmetric or asymmetric, but bilateral symmetric peripheral neuropathy is more common.

According to investigation and statistics on hospitalized diabetic patients in China in the 1990s by Chinese Medical Association, the incidence of peripheral neuropathy in the diabetic patients was 60.3%, and the most common was distal symmetric polyneuropathy and DAN. 68% of DPN patients died within five years after amputation. For those high-risk DPN patients, through screening and diagnosis in an early stage, by using an appropriate intervention method, the incidence of ulcers can be reduced by 60%, the incidence of amputation is reduced by 85%, the survival rate is greatly improved, and the life quality is improved. Therefore, it is very important to comprehensively recognize the DPN and perform early diagnosis and treatment on the DPN.

At present, medicine for clinically treating the diabetic peripheral neuropathy can only alleviate the symptoms of the patients, and cannot block the progress of the diabetic peripheral neuropathy. Therefore, there is an urgent need to develop a novel anti-diabetic peripheral neuropathy medicine capable of blocking the pathological progress of the diabetic peripheral neuropathy. The pathogenesis of the diabetic peripheral neuropathy is complicated. Hyperglycemia is a main reason causing the peripheral neuropathy. DPN is a result of joint action of a plurality of factors, including metabolic disorders, vascular injury, neurotrophic factor deficiency, cytokine abnormality, oxidative stress and immune factors. Additionally, glucose autoxidation causes formation of reactive oxidation products, resulting in cellular oxidative stress and mitochondrial dysfunction.

The DPN firstly leads to abnormality of the sensory neurons. The main pathological changes are axonal atrophy and degeneration, or even disappearance, segmental or diffuse myelin sheath shrinkage or demyelination, and Ranvier node internode length change caused by remyelination. In the DPN, nerve fiber loss is predominant and mainly occurs in an ischemia part. At present, through a large number of sural nerve biopsies and autopsies, it was discovered that diabetic patients with distal symmetric polyneuropathy still had segmental demyelination on the basis of primary Schwann cell abnormality. This pathological change was mostly found in medullated great nerves such as sural nerves, tibial nerves and median nerves, and might cause Ranvier node interval reduction, nerve conduction velocity deceleration, and symptoms such as hypaesthesia and fatigue. Therefore, it is considered that an anti-diabetic peripheral neuropathy therapeutic effect can be achieved by protecting and promoting growth of sensory neurites.

Drofenine, having a following structural formula:

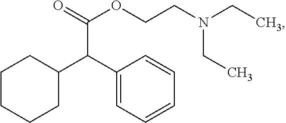 [SMILES: CCN(CC)CCOC(=O)C(c1ccccc1)C1CCCCC1]

is an effective BChE competitive inhibitor, and is clinically used for spasmolysis. Its anti-diabetic peripheral neuropathy (DPN) effect by protecting the peripheral sensory neurons and/or relieving mitochondrial dysfunction has not been reported. The present invention further discovers that the drofenine has an effect of protecting peripheral sensory neurons. Therefore, the present invention further discovers its potential on novel indication treatment.

### SUMMARY

One objective of the present invention is to develop a novel medical application of drofenine or a pharmaceutically acceptable salt thereof, i.e., application of drofenine or a pharmaceutically acceptable salt thereof to preparation of medicine for treating peripheral neuron axonal injury and peripheral related neuropathy.

Application of drofenine or a pharmaceutically acceptable salt thereof to preparation of medicine for treating peripheral neuron axonal injury and peripheral related neurodegenerative diseases is provided.

More preferably, application of drofenine or a pharmaceutically acceptable salt thereof to preparation of medicine for treating diabetic peripheral neuropathy is provided.

More preferably, application of drofenine or a pharmaceutically acceptable salt thereof to preparation of medicine for treating peripheral neuropathy caused by chemotherapy is provided.

More preferably, drofenine or a pharmaceutically acceptable salt thereof are capable of promoting growth of peripheral sensory neurites.

Application of a pharmaceutical composition containing drofenine or a pharmaceutically acceptable salt thereof to preparation of medicine for treating peripheral neuron axonal injury and peripheral related neuropathy is provided.

Application of a pharmaceutical composition to preparation of medicine for treating diabetic peripheral neuropathy is provided. The pharmaceutical composition includes drofenine or a pharmaceutically acceptable salt thereof and pharmaceutically acceptable auxiliary materials.

More preferably, the pharmaceutically acceptable salt includes an inorganic acid salt and an organic acid salt, such as malate.

According to the present invention, through a large amount of experimental screening, novel action mechanisms and pharmaceutical effects of the drofenine are found. Experiment results show that the drofenine has a good effect on protecting the peripheral sensory neurons.

The compound can be used for preparing medicine for treating diabetic peripheral neuropathy and neuropathy related to peripheral sensory neuron injury.

### BRIEF DESCRIPTION OF THE DRAWINGS

FIG. 1 is a fluorogram showing that drofenine can significantly promote growth of peripheral sensory neurites. ^{###}P<0.001, representing drofenine treated vs. control. One-way analysis of variance.

FIG. 2 is a histogram showing that the drofenine can significantly promote growth of peripheral sensory neurites.

### DESCRIPTION OF THE EMBODIMENTS

The present invention is further illustrated below with reference to specific embodiments. However, the embodiments should not be construed as limiting the present invention.

### Experimental Embodiment 1: Experiment Using Drofenine to Promote Growth of Peripheral Sensory Neurites

The present invention tested the effect of the drofenine on promoting the growth of the peripheral sensory neurites in peripheral sensory neuron DRG. The experiment showed that the drofenine has a significant effect on promoting the growth of the peripheral sensory neurites.

### 1. Experiment Principle

The experiment was based on that DPN firstly leads to abnormality of sensory neurons, and the main pathological changes were axonal atrophy and degeneration, or even disappearance. For DRG, as peripheral sensory neurons, its neurite growth state is closely related to a development process of the DPN. The inventor of the present invention added DMSO and different concentrations of drofenine into DRG primary cells for common incubation for a certain time. Then, the growth of the neurites was determined by a β-tubulin III fluorescent staining detection method, and lengths were quantified by ImageJ. The effect of the drofenine on promoting the growth of the peripheral sensory neurites was evaluated on the basis.

### 2. Experiment Materials and Method

1) β-tubulin III for fluorescent staining was purchased from Sigma company. All cell culture reagents were purchased from Gibco company. DRG cells were taken from C57BL6 mouse spines.

2) β-tubulin III staining experiment: DRG cells were inoculated in a 12-hole plate at 100 cells/hole. After the DRG cells were attached to a wall overnight, incubation was performed for 24 hours by different concentrations of drofenine. Then, an original culture medium is removed. 4% of paraformaldehyde was added into each hole for immobilization for 15 min. Permeabilization was performed for 5 min by 0.3% Triton. After PBS washing, room-temperature sealing was performed for 1 h. Primary antibody overnight incubation was performed at 4° C. (1:1000). Goat anti-mouse 1:200 room-temperature incubation in a dark place was performed for 1 h. Finally, images were observed in a fluorescence microscope.

### 3. Experiment Result

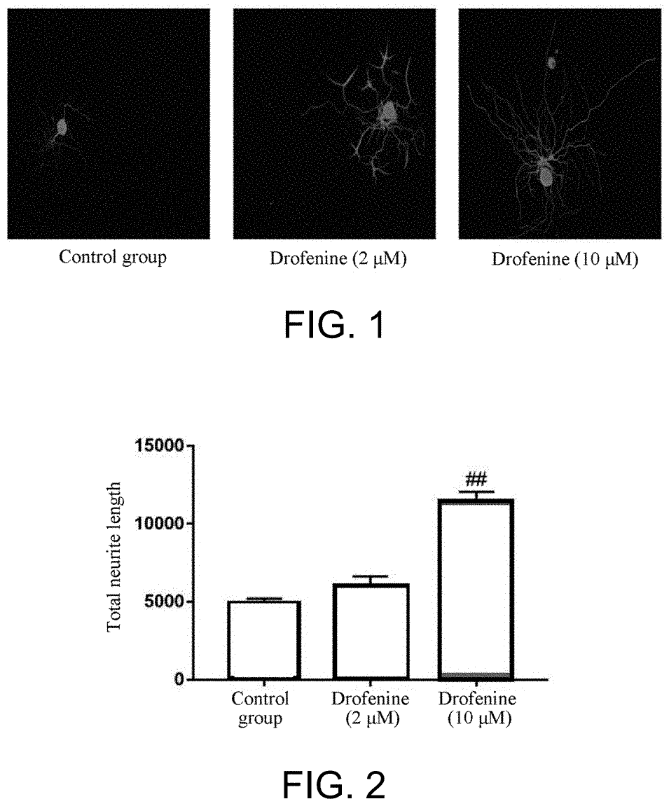

The result is as shown in FIG. 1, and the drofenine can promote the growth (fluorogram) of the peripheral sensory neurites in a concentration dependent way. As shown in FIG. 2, the drofenine can promote the growth (quantitative diagram) of the peripheral sensory neurites in a concentration dependent way, indicating that the drofenine has a significant effect on promoting the growth of the peripheral sensory neurites in a concentration dependent way.

### Embodiment 2: Experiment Using Drofenine to Relieve Motor Nerve Conduction Velocity and Sensation Loss of Diabetic Peripheral Neuropathy

The present invention tested the effect of drofenine on the motor nerve conduction velocity and pain response of the drofenine on the diabetic peripheral neuropathy on STZ-induced type I diabetic mice and db/db type II diabetic mice. The experiment showed that the drofenine has a significant effect on relieving motor nerve conduction velocity and sensation loss of the diabetic peripheral neuropathy.

### 1. Experiment Principle

The experiment was based on that when rodents received a mechanical stimulation at their paws, they generated withdrawal reflex. A Von Frey may provide stimulation force in a range of 0.008 g to 300 g. A magnitude of the stimulation force was determined by a thickness and an extending length of a nylon wire. The nylon wire with a proper thickness was selected according to practical conditions in the experiment. The proper extending length was regulated to realize vertical stimulation on the skin. The magnitude of the stimulation force might be regulated by regulating the extending length and changing the nylon wire until the nylon wire bended. The response of mice on the pain was evaluated on the basis.

### 2. Experiment Materials and Method

1) Animal grouped administration condition (type I diabetic mice): C57BL/6J mice were purchased from Beijing Vital River. After adaptive feeding for a week, intraperitoneal injection of STZ (180 mg/kg) was performed. Blood glucose was measured after 4-7 days. Mice with the blood glucose higher than 16 mmol/L were randomly selected. After 6 weeks after the STZ injection, grouping was performed according to body weights and the blood glucose to obtain a control group (non-diabetic mice), a model group (DPN mice), drofenine (10 mg/kg), and drofenine (20 mg/kg). Intraperitoneal injection of drofenine was performed for 4 weeks. The motor nerve conduction velocity, mechanical pain and heat pain were respectively tested in 0^{th} week, 4^{th} week and 8^{th} week after the STZ injection.

2) Animal grouped administration condition (type II diabetic mice): 16-week-old db/db mice were selected and subjected to adaptive feeding for a week. Grouping was performed according to body weights and blood glucose to obtain a control group, a model group, a drofenine low-dose group (10 mg/kg), and a drofenine medium-dose group (20 mg/kg). Before administration, the motor nerve conduction velocity and a 50% mechanical pain threshold were measured to determine DPN symptoms. Intraperitoneal injection of drofenine was performed for 4 weeks. The mechanical pain, heat pain and motor nerve conduction velocity were tested in each week.

2) Von Frey tactile measuring suits were purchased from a company, and a plantar test instrument was purchased from UGO company.

3) Paw withdrawal Experiment: A nylon wire with a proper thickness was selected. A proper extending length was regulated to realize vertical simulation on the skin. The magnitude of stimulation force might be regulated by regulating the extending length and changing the nylon wire until the nylon wire bended. Each mouse was tested for six times to determine a threshold. A 50% mechanical pain threshold of the mice was calculated according to 50% threshold=(10\hat{ } (x^{f}+kδ))/10000.

4) Heat pain testing experiment: An infrared instrument was placed under the sole of the mouse, and kept still. The time was recorded when the paw of the mouse withdrew. This time was heat pain response time of the mouse.

5) Motor nerve conduction velocity testing experiment: (1) Electrode placement: A stimulation electrode was placed at a nerve trunk, a recording electrode was placed at a muscle belly, a reference electrode was placed at a muscle tendon, and a ground wire was placed between the stimulation electrode and the recording electrode. (2) Motor nerve conduction velocity calculation: Super-strong stimulation was performed on distal and proximal ends of the nerve trunk, 2 compound muscle action potentials might be recorded on muscles dominated by this nerve, and different latency periods thereof were measured. The nerve conduction velocity was obtained by dividing a distance between the distal and proximal ends by a latency period difference between two points. A calculation formula was: Nerve conduction velocity (m/s)=distance between two points (cm)×10/latency period difference between two points.

### 3. Experiment Result

The result is as shown in Tables 1-3, and the drofenine can significantly relieve motor nerve conduction velocity and sensation loss of the type I diabetic peripheral neuropathy. ***P<0.001, representing DPN vs. control. ^{###}P<0.001, representing drofenine treated vs. DPN. One-way analysis of variance.

TABLE 1
Motor nerve conduction velocity (mean ± SEM., n = 6, m/s)

| Number of | | | | |
|---|---|---|---|---|
| adminis- | | | | |
| tration | Control | Model | Drofenine | Drofenine |
| weeks | group | group | (10 mg/kg) | (20 mg/kg) |
| 0 | 53.45 ± 3.04 | 40.13 ± 1.20** | 39.61 ± 1.06 | 40.63 ± 1.03 |
| 2 | 52.28 ± 2.28 | 37.61 ± 2.32** | 40.55 ± 2.14 | 43.82 ± 1.37# |

**P < 0.01.
***p < 0.001 vs control group.
#p < 0.05.
##p < 0.01.
###p < 0.001 vs model group.

TABLE 2
50% paw withdrawal threshold (mean ± SEM., n = 6, g)

| Number of | | | | |
|---|---|---|---|---|
| administration | Control | Model | Drofenine | Drofenine |
| weeks | group | group | (10 mg/kg) | (20 mg/kg) |
| 0 | 1.41 ± 0.15 | 2.77 ± 0.19*** | 2.77 ± 0.19 | 2.77 ± 0.19 |
| 1 | 1.41 ± 0.10 | 3.03 ± 0.24*** | 2.53 ± 0.25 | 2.06 ± 0.31 |
| 2 | 1.43 ± 0.14 | 2.96 ± 0.13*** | 1.31 ± 0.13### | 1.31 ± 0.13### |
| 3 | 1.31 ± 0.09 | 2.87 ± 0.00*** | 1.40 ± 0.13### | 1.27 ± 0.12### |
| 4 | 1.34 ± 0.07 | 2.87 ± 0.00*** | 1.45 ± 0.11### | 1.35 ± 0.10### |

**P < 0.01.
***p < 0.001 vs control group.
#p < 0.05.
##p < 0.01.
###p < 0.001 vs model group.

TABLE 3
Heat response (mean ± SEM., n = 6, s)

| Number of | | | | |
|---|---|---|---|---|
| administration | Control | Model | Drofenine | Drofenine |
| weeks | group | group | (10 mg/kg) | (20 mg/kg) |
| 0 | 7.30 ± 0.44 | 10.82 ± 0.63*** | 11.43 ± 0.98 | 10.3 ± 0.60 |
| 1 | 7.38 ± 0.56 | 11.76 ± 0.98*** | 11.12 ± 0.66 | 10.77 ± 0.94 |
| 2 | 7.38 ± 0.61 | 11.66 ± 0.62*** | 10.28 ± 0.68 | 8.64 ± 0.69## |
| 3 | 7.32 ± 0.50 | 11.73 ± 0.80*** | 8.96 ± 0.26## | 7.91 ± 0.54## |
| 4 | 7.34 ± 0.51 | 11.63 ± 0.79*** | 8.78 ± 0.26## | 7.71 ± 0.68## |

**P < 0.01.
***p < 0.001 vs control group.
#p < 0.05.
##p < 0.01.
###p < 0.001 vs model group.

As shown in Tables 4-6, the drofenine can significantly relieve motor nerve conduction velocity and sensation loss of db/db type II diabetic peripheral neuropathy. ***P<0.001, representing db/db vs. db/n. ###P<0.001, representing drofenine treated vs. db/db. One-way analysis of variance.

TABLE 4
Motor nerve conduction velocity (mean ± SEM., n = 6, m/s)

| Number of | | | | |
|---|---|---|---|---|
| administration | | | Drofenine | Drofenine |
| weeks | db/n | db/db | (10 mg/kg) | (20 mg/kg) |
| 0 | 43.45 ± 3.04 | 35.13 ± 2.21** | 34.61 ± 1.06 | 35.63 ± 1.03 |
| 1 | 45.28 ± 4.28 | 34.61 ± 2.32*** | 36.55 ± 2.14 | 38.82 ± 1.37 |
| 2 | 47.12 ± 3.03 | 30.32 ± 3.01*** | 38.23 ± 3.20## | 40.23 ± 2.02## |
| 3 | 46.22 ± 3.13 | 32.35 ± 2.32*** | 39.34 ± 2.89## | 41.56 ± 2.35## |
| 4 | 46.28 ± 3.30 | 33.26 ± 3.33*** | 40.12 ± 2.87## | 42.28 ± 2.26## |

**P < 0.01.
***p < 0.001 vs db/n.
#p < 0.05.
##p < 0.01.
###p < 0.001 vs db/db.

TABLE 5
50% paw withdrawal threshold (mean ± SEM., n = 6, g)

| Number of | | | | |
|---|---|---|---|---|
| administration | | | Drofenine | Drofenine |
| weeks | db/n | db/db | (10 mg/kg) | (20 mg/kg) |
| 0 | 1.81 ± 0.21 | 2.83 ± 0.15*** | 2.81 ± 0.17 | 2.83 ± 0.19 |
| 1 | 1.62 ± 0.12 | 3.16 ± 0.10*** | 2.82 ± 0.22 | 2.62 ± 0.26 |
| 2 | 1.77 ± 0.34 | 3.02 ± 0.14*** | 2.21 ± 0.13## | 2.01 ± 0.13## |
| 3 | 1.63 ± 0.22 | 3.11 ± 0.08*** | 2.09 ± 0.34## | 1.92 ± 0.12## |
| 4 | 1.62 ± 0.11 | 2.90 ± 0.07*** | 1.98 ± 0.21## | 1.82 ± 0.01## |

**P < 0.01.
***p < 0.001 vs db/n.
#p < 0.05.
##p < 0.01.
###p < 0.001 vs db/db.

TABLE 6
Heat response (mean ± SEM., n = 6, s)

| Number of | | | | |
|---|---|---|---|---|
| administration | | | Drofenine | Drofenine |
| weeks | db/n | db/db | (10 mg/kg) | (20 mg/kg) |
| 0 | 6.52 ± 0.44 | 10.82 ± 1.63*** | 10.43 ± 0.98 | 10.39 ± 0.30 |
| 1 | 8.51 ± 0.53 | 14.76 ± 0.98*** | 13.52 ± 0.69 | 10.77 ± 0.54## |
| 2 | 7.58 ± 0.51 | 12.56 ± 1.22*** | 10.58 ± 0.32## | 8.54 ± 0.79## |
| 3 | 7.52 ± 0.32 | 15.03 ± 0.90*** | 8.96 ± 0.56## | 8.51 ± 0.55## |
| 4 | 7.54 ± 0.51 | 14.53 ± 0.79*** | 9.78 ± 0.56## | 8.01 ± 0.68## |

**P < 0.01.
***p < 0.001 vs db/n.
#p < 0.05.
##p < 0.01.
###p < 0.001 vs db/db.

The foregoing embodiments are merely exemplary embodiments of the present invention, but are not intended to limit the present invention. The protection scope of the present invention is limited by the claims. A person skilled in the art may make various modifications or equivalent replacements to the present invention within the essence and protection scope of the present invention. The modifications or equivalent replacements shall also fall within the protection scope of the present invention.

1. Application of drofenine or a pharmaceutically acceptable salt thereof in the preparation of medicine for treating peripheral neuron axonal injury and peripheral related neuropathy.

2. Application of drofenine or a pharmaceutically acceptable salt thereof in the preparation of medicine for treating peripheral neuron axonal injury and peripheral related neurodegenerative diseases.

3. Application of drofenine or a pharmaceutically acceptable salt thereof in the preparation of medicine for treating diabetic peripheral neuropathy.

4. Application of drofenine or a pharmaceutically acceptable salt thereof in the preparation of medicine for treating peripheral neuropathy caused by chemotherapy.

5. The application according to claim 1, wherein the drofenine or the pharmaceutically acceptable salt thereof are capable of promoting growth of peripheral sensory neurites.

6. Application of a pharmaceutical composition containing drofenine or a pharmaceutically acceptable salt thereof in the preparation of medicine for treating peripheral neuron axonal injury and peripheral related neuropathy.

7. Application of a pharmaceutical composition in the preparation of medicine for treating diabetic peripheral neuropathy, wherein the pharmaceutical composition comprises drofenine or a pharmaceutically acceptable salt thereof and pharmaceutically acceptable auxiliary materials.

8. The method according to claim 2, wherein the drofenine or the pharmaceutically acceptable salt thereof are capable of promoting growth of peripheral sensory neurites.
